(19) United States
(12) Patent Application Publication
(10) Pub. No.: US 2013/0344051 A1
(43) Pub. Date: Dec. 26, 2013

(54) Compostion of matter for treating or preventing various types of cancer and neoplastic diseases in a mammal

(71) Applicants: BENEDICT SCHUE LIAO, MONTEREY PARK, CA (US); AUSTIN LEWIS LIAO, FULLERTON, CA (US); JUDY FU-CHUAN LI-LIAO, FULLERTON, CA (US); BURTON ARTHUR LI-LIAO, WALNUT, CA (US); JUSTIN LIAO, IRVINE, CA (US); EDWARD REGEAN LIAO, IRVINE, CA (US); ROBERT LIAO, LOS ANGELES, CA (US); SUSAN JOAN LIAO-NIENG, CHICAGO, IL (US); SU-LIEN LIAO-CHEN, BALTIMORE, MD (US); LUDWIG SCHUE-LIAO, BALTIMORE, MD (US); LILY LIAO, LOS ANGELES, CA (US); SUSAN SU-HSIN LIAO-TUNG, IOWA CITY, IA (US); ALEX BISMARK LIAO, LOS ANGELES, CA (US); STANLEY LEO, LAS VEGAS, CA (US); NELSON BENSON LEO, LAS VEGAS, NV (US)

(72) Inventors: BENEDICT SCHUE LIAO, MONTEREY PARK, CA (US); AUSTIN LEWIS LIAO, FULLERTON, CA (US); JUDY FU-CHUAN LI-LIAO, FULLERTON, CA (US); BURTON ARTHUR LI-LIAO, WALNUT, CA (US); JUSTIN LIAO, IRVINE, CA (US); EDWARD REGEAN LIAO, IRVINE, CA (US); ROBERT LIAO, LOS ANGELES, CA (US); SUSAN JOAN LIAO-NIENG, CHICAGO, IL (US); SU-LIEN LIAO-CHEN, BALTIMORE, MD (US); LUDWIG SCHUE-LIAO, BALTIMORE, MD (US); LILY LIAO, LOS ANGELES, CA (US); SUSAN SU-HSIN LIAO-TUNG, IOWA CITY, IA (US); ALEX BISMARK LIAO, LOS ANGELES, CA (US); STANLEY LEO, LAS VEGAS, CA (US); NELSON BENSON LEO, LAS VEGAS, NV (US)

(73) Assignee: GARFIELD MEDICAL GROUP, MONTEREY PARK, CA (US)

(21) Appl. No.: 14/012,629

(22) Filed: Aug. 28, 2013

Related U.S. Application Data
(63) Continuation of application No. 13/374,328, filed on Dec. 16, 2011.

(51) Int. Cl. A61K 38/48; A61K 31/375

(52) U.S. Cl. CPC A61K 38/4873; A61K 31/375
USPC 424/94.3; 435/188

(57) ABSTRACT

A composition of matter can reduce size of cancerous tumors in mammals. The composition of matter comprises Bromelain infused with vitamin C creating a protein enzyme complex which can be administered in an effective amount to reduce the size of cancerous tumors in mammals.

### CROSS REFERENCE TO RELATED APPLICATIONS

This application is a continuation of U.S. patent application Ser. No. 13,374,328 filed on Dec. 16, 2011 which is incorporated by reference in its entirety.

### FIELD OF THE INVENTION

This invention relates compositions of matter that demass cancerous tumors.

### BACKGROUND OF THE INVENTION

According to the recent studies and reports, various types of cancer and neoplastic diseases have been amount the number one cause of deaths in America.

The search for new medicines with non-toxic, low side effects which are compatible with most other drugs have been under way since Cohen in 1964, Renzini in 1972, and Tinozzi in 1978.

Bromelain was first isolated from pineapple fruit in 1891 by Marcano. (Marcano, Bull. Pharma. 5, 77 (1891)). Briefly, the pineapple is a tropical plant with edible multiple fruit consisting of coalesced berries, and the most economically significant plant in the Bromeliaceae family. Pineapples may be cultivated from a crown cutting of the fruit, possibly flowering in 20-24 months and fruiting in the following six months. Pineapple does not ripen significantly post-harvest. Heinecke and Gortner discovered stem bromelain, as a new proteinase preparation from pineapple plant in 1957, by precipitating with acetone and with ammonium sulfate in 1961. Further purification of crude preparation was formulated by Gibian and Bratfish in 1960 and a patent right was granted to Pineapple Research Institute (U.S. Pat. No. 3,002,891) and to AG Schering Company (U.S. Pat. No. 2,950,227). Bayer T., reported the anti-inflammatory effects in flavonoids (Phytochemistry, 28, pp 2373-2378 (1989) in 1989.

Utilization of plant and/or fruit flavonoids such as Rutin, Quercetin, Naringenin and Hesperetin in inhibiting tumor growth have been reported by Saija A., Scalese M. et al., (Free Radical Biology and Medicine vol. 19, no.4 Pp 481-486 (1995)), Felicia, V. S.; Najla Guthrie, et al. depicted evidence of inhibition of human breast cancer cell proliferation and delay of mammary tumor-genesis by flavonoids and citrus juices. (Nutrition and Cancer vol. 26, no. 2, pp 167-181 (1996)).

In the year 2000, Revilla, E., and Ryan J. M., et al. analyzed of several phenolic compounds with potential antioxidant properties in grape extracts and wines by high-performance liquid chromatography-photodiode array detection without sample preparation. (Journal of Chromatographia; 6, 881 (1-2); pp 461-469 (2000)).

Also in 1991 by Kandaswai, C. and Perkins E. et al reported that citrus flavonoids have anti-proliferative effects on human squamous cell carcinoma in vitro: (Cancer Lett.; 56, pp. 147-152 (1991)). Guthrie, N., and Moffatt, M., et al claimed Naringenin, a flavonoid from grapefruit, has anti-proliferative effect in human breast cancer cell lines. (National Forum Breast Cancer, Montreal, p. 119 (1993)).

The study of Rakotoarison, D A. et al showed anti-oxidant activity of polyphenolic extracts from flowers of Crataegus Monogyna (Pharmazie: 52: Pp 60-64 (1997)). Kaul, T N. and Elliott Middleton et al also reported the anti-viral effect of Citrus flavonoids on human viruses. (Journal of Medical Virology: 15; Pp 71-79 (1985)). It has also been reported that pant bio-flavonoids exhibit various biochemical and pharmacological activities including anti-oxidant, anti-inflammatory, anti-cancer, anti-viral and anti-platelet aggregation.

Nair, M G; and Wang, H B. et al reported that a method of inhibiting cyclooxygenase and inflammation using cyaniding, Ranelleti, F. O., and Ricci R. et al, reported growth inhibitory effect of Quercetin and presence of type 11 estrogen binding sites in human colon cancer cell lines and primary colo-rectal tumors; (International Journal of Cancer: 50; pp 486-492 (1992)), Scambia, G. and Ranelleti, F. O. et al; reported that Quercetin inhibits the growth of a multi-drug-resistant estrogen receptor—negative MCF-7 human breast cancer cell line expressing type-II estrogen-binding sites: (Cancer Chemotherapy, Pharmacology: 28, pp 255-258 (1991)) also reported in Type II16 estrogen binding sites in a Iymphoblast cell line and growth inhibiting effect of estrogen, anti-estrogen and bioflavonoids; (International Journal of Cancer: 46 pp 1112-1116 (1990)), Castillo, M. H. and Perkins, E., et al also reported the effect of the bioflavonoid-Quercetin on squamous cell carcinoma of head and neck origin; (American Journal of Surgery: 158, pp 351-355 (1989)).

Zhao, J., Wang, J., et al published a study showing anti-tumor promoting activity of a polyphenolic fraction isolated from grape seeds and identification of Procyanidin B 5-3′-gallate as the most effective antioxidant constituent; (Carcinogenesis, September; 20(9); pp 1737-1745 (1999)).

In 1995 Harrach T., Eckert K., Schulze-Forster K. and Maurer H. Rainer et al reported isolation and partial characterization of basic proteinases from stem bromelain. (Journal Protein Chemistry 14: pp 41-52, 1995), and Again in 1997 they reported isolation and characterization of two forms of an acidic bromelain stem proteinase. (Journal Protein Chemistry 20: pp 53-64, 1997).

In 1985 Taussig, and Batkin et al suggested that the enzyme complex from Ananas Comosus (pineapple) produced inhibition of tumor growth in vitro (Planta Med. 6: 538-539, 1985). and outlined its clinical application, (Journal Ethnopharmacology: 22, P 191-203, 1988). The same enzyme complex also produces platelet anti-aggregation , and fibrinolytic activity, (reported by Marz, in 1982, Heinecke in 1972, Heinecke and Yokoyama in 1957). In 1979 Klaue, Amen, and Roman et al reported use of bromelain as chemical debridement agent in 3rd degree burn patients. (European Surgical research, 1979, PP 355-359). In same year of 1988 the same team also discovered that Bromelain has an anti-metastatic effect with or without its proteolytic and anti-coagulant activity, (Journal of Cancer Research Clinical Oncoloy, 114: pp 507-508, 1988).

The prior art includes U.S. Pat. No. 5,928,640 issued to Mynott; U.S. Pat. No. 6,803,038 issued to Mauer; and U.S. Patent Application 2009/0017024 filed by Estok.

Estok teaches composition of matter that can reduce tumor size comprising tablets made from Bromelain using vitamin E with starch or lactose. Estok lists using Vitamin E as a “useful dosage form” but not to “acidify” the compound. Rather, Estok acidifies the compound by adding one of several dozen salts and acids, though not Vitamin C (Vitamin C includes L-ascorbic acid, ascorbate, or dehydroascorbic acid, which are understood to be equivalent). The others use the word “bromelain,” but do not consider making tablets with starch or lactose or adding vitamin C. The chemical structures are simply different to solve a different problem.

The present inventors have discovered that the extract of Bromelain proteins which derived from the fruit and stem of ananas comosus (pineapple). Bromelian, Ananase, comosain, lnflamen, Extranase, Traumanase which are effective in treating and/or preventing various types of cancer and neoplastic diseases including breast, colon, lung, ovarian, cervical, and uterine cancers, the method comprises administered an effective amount of bromelainase and its extract from Anasas Comosus. By the following attributes: Anti-inflammtory properties; Anti-platelet aggregation (Mynott et al in 1998, suggested that it changes the tumor surface antigen thus preventing tumor cells from attacking the normal tissues); Fbrinolytic properties; Anti-tumor genesis, this action was probably due to release of tumor necrotizing factors (TNF) in T-cells of WBC. Taussig etal in 1985 and Taussig, and Batkin et al in 1988 both indicated that Bromelain extract can be used in inhibiting tumor growth, the T-cells, peripheral blood mononuclear lymphocytes (PBMN) with influence of bromelain, they produce and outpour of CD2, CD3, Interlukin IB, ll-6, ll-8, and TNF. and attack the tumor surface antigens of CD44, CD45, CD47. Anti-dedifferentiation both in vitro and in vivo in cancer cell lines, in animal, and human experiments.

### BRIEF SUMMARY OF THE INVENTION

A composition of matter can reduce size of cancerous tumors in mammals. The composition of matter comprises Bromelain infused with vitamin C creating a protein enzyme complex which can be administered in an effective amount to reduce the size of cancerous tumors in mammals.

### BRIEF DESCRIPTION OF THE SEVERAL VIEWS OF THE DRAWINGS

Having thus described the invention in general terms, reference will now be made to the accompanying drawings, which are not necessarily drawn to scale, and wherein:

FIG. 1 is a schematic process of Bromelain extraction procedure from ananas comosus.

FIG. 2 is a schematic process of Bromelain precipitation by acetone.

FIG. 3 is a schematic process of Bromelain precipitation by ammonium sulfate.

FIG. 4 is a schematic process of Bromelain precipitation by dialysis.

FIG. 5 is a schematic process of oral administration of Bromelain.

FIG. 6 is a schematic process of intravenous administration of Bromelain.

FIG. 7 shows the unexpected results of the present invention demassing tumors in human subjects.

### DETAILED DESCRIPTION OF THE INVENTION

Embodiments of the present invention overcome many of the obstacles associated with reducing the size of cancerous tumors, and now will be described more fully hereinafter with reference to the accompanying drawings that show some, but not all embodiments of the claimed inventions. Indeed, the invention may be embodied in many different forms and should not be construed as limited to the embodiments set forth herein. Rather, these embodiments are provided so that this disclosure will satisfy applicable legal requirements. Like numbers refer to like elements throughout.

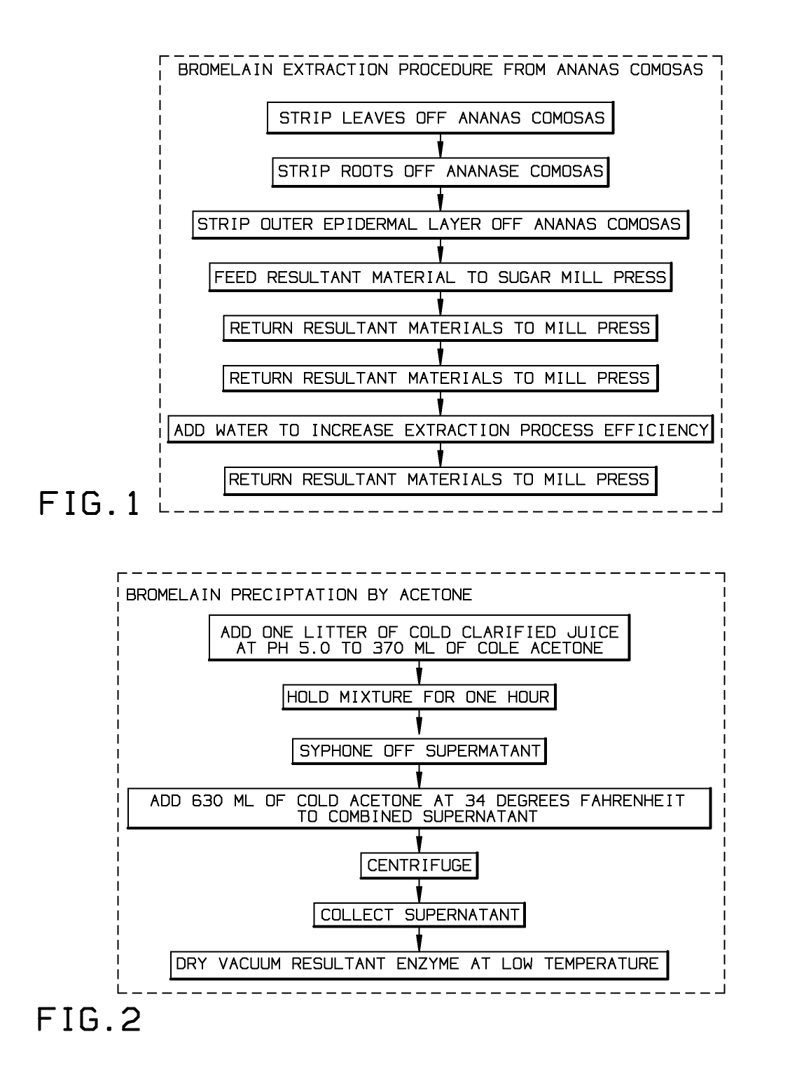

FIG. 1 shows a Bromelain extraction procedure from ananas comosus (the pineapple). Pineapple stumps were stripped of leaves and roots and outer epidermal layers. The pineapple stumps were then cleaned and fed to a sugar mill press, the residues were returned to the mill press for an additional three times, after the second run, water was added to increase the efficiency of the extraction process. The juice produced was about 35% of peeled pineapple stumps. The protein produced was 150 grams per 10 pounds of peeled stumps. The total yield of proteinases, expressed in milk clotting units (M.C.U.) was 25,000 units per pound of stump.

FIG. 2 shows Bromelain precipitation by acetone. One liter of cold clarified stem juice at pH 5.0 was added to 370 ml of cold acetone. As used here “cold” means at a temperature of 34 degrees Fahrenheit. The mixture was held for an hour and the supernatant containing the desired enzyme substances was syphoned off. The precipitate contained about one-sixth of the activity of the final product. After all assays on this fraction were run, this fraction was discarded. Again 630 ml of cold acetone at 34 degree F. was added to a combined supernatant, the precipitate was collected by centrifuging of the combination supernatant. The enzyme was left in the centrifuge bottle and dried in a vacuum chamber at a low temperature. The yield of enzyme was 9.5 grams of a white powder which contained 4,500 M.C.U./per gram (which is about 20,655 M.C.U. per pound) based on the activity of the starting juice, which represented 40% of the proteolytic activity originally present in the juice.

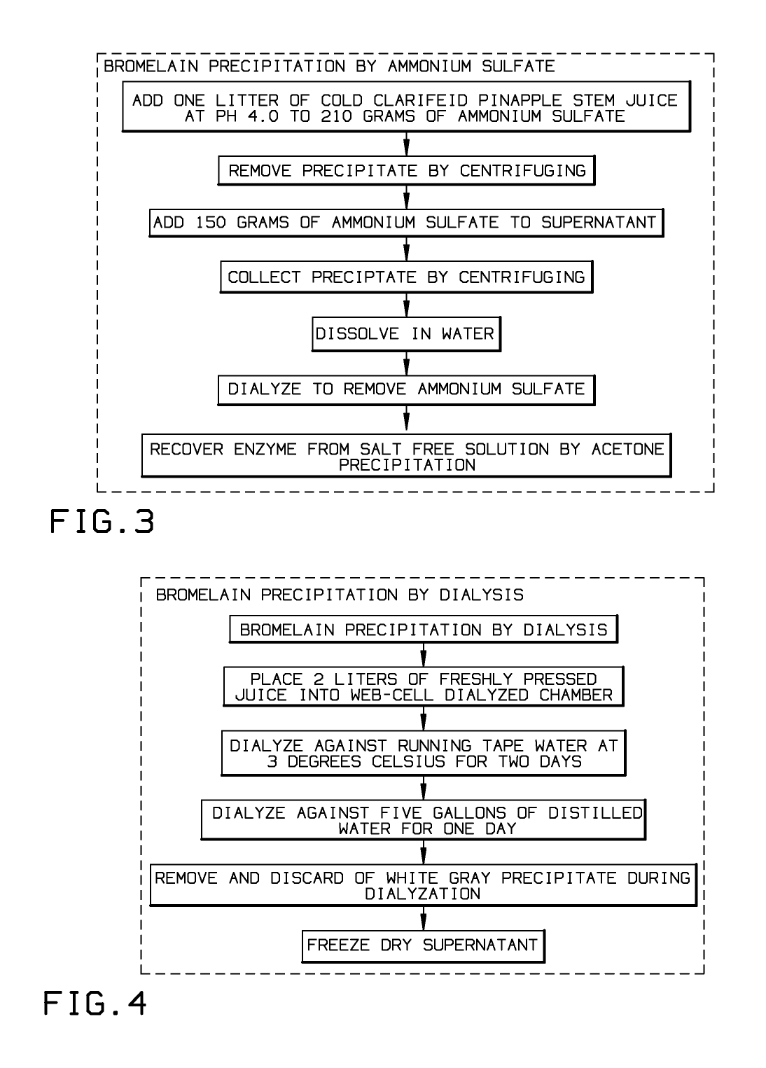

FIG. 3 shows Bromelain precipitation by ammonium sulfate. 1000 ml of cold clarified pineapple stem juice at PH of 4.0 were added 210 gram of ammonium sulfate. As used here “cold” means at a temperature of 40 degrees Fahrenheit. The precipitate formed was removed by centrifuging. Another 150 gram of ammonium sulfate was added to the supernatant. The precipitate was collected by centrifuging. Then dissolved in water and dialyzed to remove the ammonium sulfate. The enzyme was recovered from the salt free solution by acetone precipitation. The total yield was 115 mg of precipitates of protein with activity of 2,200 M.C.U./gram.

FIG. 4 demonstrates Bromelain precipitation by dialysis. 2000 ml of the freshly pressed juice was placed in a web-cell dialyzed chamber, and was dialyzed against running tap water at 3 degrees Centigrade for two days and then dialyzed against five gallons of distilled water for one day. During the dialysis a light grey precipitate appeared was removed and discarded. The supernatant was then freeze dried and yield of 9.6 grams of 4200 M.C.U. enzyme per liter.

FIG. 5 describes how Bromelainases can take Bromelain (also known as anase, comosain, inflamen, extranse and traumanase) herein an “active ingredient” and be applied orally to a patient. Three examples are provided for hard and/or soft gelatin capsules are prepared with ingredients as follows: Formulation-1: 500 mg of active ingredient are combined with 200 mg of Vitamin C and 50 mg of a starch or lactose carrier to create a 750 mg capsule. Formulation-2: 1000 mg of active ingredient are combined with 300 mg of Vitamin C and 200 mg of a starch or lactose carrier to create a 1500 mg capsule. Formulation-3: 1500 mg of active ingredient are combined with 300 mg of Vitamin C and 100 mg of a starch or lactose carrier to create a 1900 mg capsule. This is known as an “effective amount.” Bromelain infused with vitamin C creatiese a protein enzyme complex which can be administered in an effective amount to reduce the size of cancerous tumors in mammals.

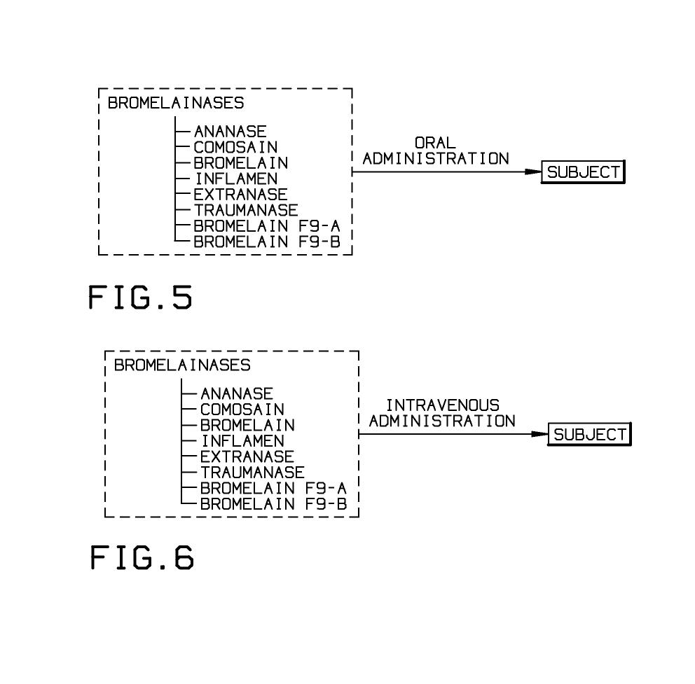

FIG. 6 describes how Bromelainases can take Bromelain (also known as anase, comosain, inflamen, extranse and traumanase) herein an “active ingredient” and be applied intravenously to a patient. Dosage should be 50 to 60 mg of active ingredient per kilogram of body weight for body weight of 50 to 60 kg, which equivalent of 2000 to 3000 mg each week/twice a week. This is known as an “effective amount.” Bromelain should be administered intravenously in a 3 to 4 hour period for 12 weeks to 2 years depending on the patient as noted in the examples below.

### EXAMPLE 1

### Toxicity of Bromelain in Mice by Oral Administration

As noted in the background section, one difficulty of prior art chemotherapy is toxicity of the compound in humans. Of course, other mammals also have adverse reaction to toxins. Accordingly, testing the toxicity of Bromelain in mice by oral administration is necessary to discern if a treatment regimen is toxic. The present invention solved that problem in the following manner: 30 specimens of 8 week old, with specific pathogen free, Imprinting Control Region (ICR) female mice, each weighing 25-30 grams, were divided into five groups with six mice in each cage and were kept in separated cages under an environment of 23±3 degrees Centigrade, relative humidity of 45±5%, and 12 hours light/12 hours dark photoperiod. The mice were fed with Harlan Teklad-2018 global rodent diet (18% protein) and drinking water was sterilized.

Bromelain was dissolved in 0.5% of purified nonionic stabilized detergent such as that marketed under the trademark TWEEN-80® to a final concentration of 50 mg/ml, 100 mg/ml, 125 mg/ml and 150 mg/ml respectively, and was orally fed to the four separated groups of mice in an amount of 0.2 ml per 20 gram of mouse body weight, that is contains 500 mg/kg, 1000 mg/kg, 1250 mg/kg, and 1500 mg/kg separately.

One group of six mice was kept as control group and was not fed the Bromelain solution. The solution was administered once every 6 months, and the mice were observed for 18 months for the signs of adverse effects or death according the following schedule: 1 hour, 4 hours, 8 hours, 12 hours, after administration and then every 12 hours thereafter. The daily weight of each mouse was recorded. On day 546, (18 months later) the mice were sacrificed, and the internal organs including liver, kidney, heart, lung, muscle, stomach, urinary bladder, intestines, pancreas, and spleen were examined visually and microscopically.

All mice were alive at 18 months, and no body weight loss occurred during this period of observation. The mice did not develop any pathological abnormality either visually or microscopically. Therefore, Bromelain extract from pineapple is not toxic when orally administered to a mammal.

### EXAMPLE 2

### Bromelain Inhibits Cancer Cell Growth In Vitro

Cancer cell lines were developed either from direct harvest from surgical specimens during surgery or from friends at different oncology institutes. The specimens were emulsified in normal saline and filtrated three times with the mesh permitting less than 5 μm particles to pass through. The supernatants were preserved in a Complete Growth Medium (CGM)) in the 75 ml flasks at 8 degree Centigrade until they are ready to be used for seeding in a standard tissue culture.

The CGM consisting of: Dulbecco's modified Eagle's medium (DMEM) supplemented with 10% heat inactivated new born calf serum, and 2% L-glutamine, penicillin (100 IU/ml), streptomycin (5 mg/ml), and neomycin (10 mg/ml) and incubated at 37 degrees Centigrade with humidified atmosphere of 90% air, 10% CO₂ and cell split at 1:2 rate twice weekly.

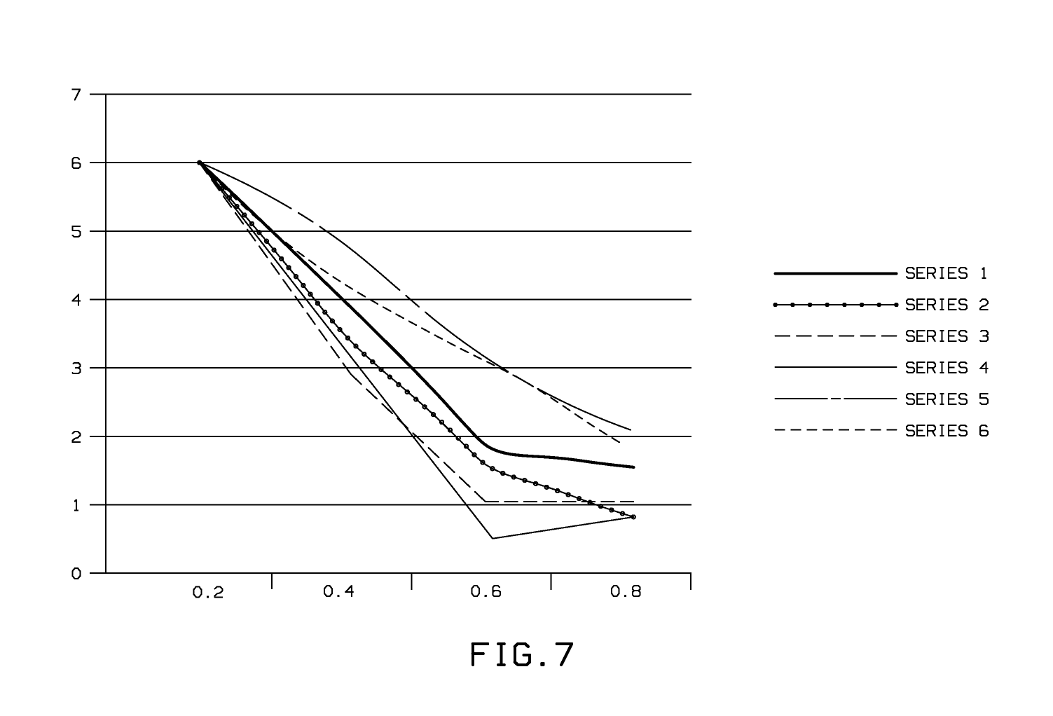

As shown in FIG. 7, six various types of cancer cell lines which included breast (series 1), colon (series 2), lung (series 3), ovarian (series 4), cervical (series 5) and uterine (series 6) cancer were used to test the sensitivity of the growth inhibition by the Bromelain in different concentrations. The cells were seeded into 96 wells tissue culture microtiter plates. The cells were maintained in a standard tissue culture CGM. Bromelain solutions were added to culture media in the following concentration: 0.2 mg/ml, 0.4 mg/ml, 0.6 mg/ml, and 0.8 mg/ml for 72 hours and tumor cells were counted with a Coulter counter. The tumor cell growth inhibition percentages are depicted in FIG. 7, which demonstrates that the growth of all cancer cell lines were inhibited in higher concentrations of Bromelain.

### EXAMPLE 3

### Bromelain Intraperitoneal Administration to Experimental Animal

Fourteen experimental animals comprising four to six weeks old white rabbits each weighting 1 to 1½ pounds were fed under a condition of 23±3 degrees Centigrade, relative humidity of 45±5% and photoperiod of 12 hours of light and 12 hours of darkness. The rabbits were divided into seven groups with 2 heads each and were fed with Harlan-Taklad rabbit diet TD-1376 containing moisture 12%, crude protein 16%, crude fat 2%, crude fiber 15%, ash 8%, nitrogen free substances 47%.

The rabbits were fed for 3 weeks with free access to the diet and water. Body weight was recorded every 7 days, and records were analyzed. All rabbits showed a normal growth rate with no significant differences among the seven groups in regard to the diet ingestion amount or the body weight gain.

The cancer cell lines were injected into six groups of the rabbits, that is two heads in each group while two heads served as control (without tumor cells injection). The cancer cell lines were developed from Example 2 above. Each head was injected 0.5 ml of different cell line fluid intraperitoneally, prefer in the peritoneum layer, then the rabbits were fed the same diet for 3-4 weeks until a tumor grew in the peritoneum. The size and location of the tumors were recorded.

When the tumors reached to 3-5 mm diameter in size. Bromelain in the amount of 25.0 mg/ml in normal saline with 100 mg of vitamin C (to keep the solution acidified), one ml of Bromelain was injected into six different group of rabbits, the Bromelain were given twice a week for 8 weeks. Notably, the Vitamin C also increased the absorbency of Bromelain and the effectiveness of the compound. This teaches away from Estok, whose misplaced reliance on Vitamin C renders that compound comparatively ineffective.

After 8 weeks of treatment, the rabbits were anesthetized with injections of ketamine 75 mg/kg in the femoral muscle and sacrificed. Blood samples were collected from the heart of each rabbit to determine the blood analysis consisting of: complete blood count (CBC), Chemistry-7 and 24, (including Liver and renal function tests), lipid profiles (including Total cholesterol, HDL, LDL, VLDL, and triglycerides), coagulation factors consisting of; prothrombin time (PT), partial thromboplastin time (PPT), and immune-globulin-E.

All the laboratory tests were analyzed, and showed no differences among or within each groups. All laboratory blood analysis were performed on rabbits of all 7 groups, The results were tested using student t-test and Microsoft Excel-7 programs. The results are depicted in the table below.

| Group | TC | TRG | HDL | SGOT | SGPT | GGTP | WBC | Hb |
|---|---|---|---|---|---|---|---|---|
| Control | 183.3 ± | 110 ± | 45.6 ± | 38.6 ± | 62.5 ± | 8 ± 2.4 | 6.8 ± | 12.3 ± |
| | 50.2 mg/dl | 40.6 mg/dl | 20.4 mg/dl | 6.2 u/I | 6.5 u/I | u/I | 2.0 k/uI | 2.2 gm/dl |
| Bromelain | 175.6 ± | 92.6 ± | 43.6 ± | 110.8 ± | 71.2 ± | 7 ± 1 | 7.3 ± 2.2 | 11.9 ± 1.9 |
| | 36.8 mg/dl | 38.8 mg/dl | 16.5 mg/dl | 30.7 u/I | 3.8 u/I | | 2 k/ul | gm/dl |

As used in the table above, TC is Total Cholesterol; TRG is Triglycerides; WBC is White Blood Cell count; HDL is High Density Lipoprotein; SGOT is Serum Glutamo-Oxalic Transferase; SGPT is Serum Glutamo-Pyruvic Transferase; and Hb is Hemoglobin.

The internal organs from the rabbits scarified in this sample including lung,heart, liver, kidney, muscle, omentum, intestine, stomach bladder and pancreas were visual examined and showed no abnormalities. One half of each organ was frozen and the other half was fixed with 10% neutral buffered formalin for 24 hours. Then the fixed organs were washed with tap waterand stepwise dehydrated with 70%, 80%, 90%, 100% ethanol and then embedded in paraffinby using Shandon-Histocentre-2. The embedded organ blocks were sectioned in 4μ thickness with a microtome and stained with hematoxylin and eosin stain. The stained specimens were made transparent with xylene, and mounted with permount on microslides.

There were no pathological abnormalities or lesions under microscopic examination. All the specimens collected from six groups of rabbits showed no evidence of persistent disease or cancer cells. Therefore, we conclude that Bromelain can serve as a chemotherapeutic agent in various types of cancer in experimental animals without side effects.

### EXAMPLE 4

### Bromelain Oral Administration to Inhibit Cancer Growth in Humans

Twenty four volunteers were divided into six groups (four in each group) and six persons serve as control group (no Bromelain treatment). All were in their 4^{th} and 6th decades with various types of cancers including breast, lung, colon, ovarian, cervical and uterine origins. All were in either Stage lll or Stage IV (the cancers had metastased widely either to lung, liver, bladder or rectum). All had been treated with either radiation or chemotherapy after surgery but experienced no positive results. The bromelainase were administered in doses of 20-30 mg/kg (or 75 to 100 Gelatin Dissolving Units (GDU) per day) based on 50-60 kg of body weight. That is 1000 mg-to-1500 mg/day (or 5,000 to 6,000 GDU/day) divided into two doses. Patients were monitored with bi-weekly blood tests consisting complete blood counts, chemistry-7, chemistry-24, kidney and liver function tests, tumor markers, coagulation factors, and X-ray or CT scan in appropriate areas to determine the size of the tumors. There were no abnormalities in all the blood tests, no anemia, no leucopenia, no thromcytopenia, no abnormal kidney nor liver function tests. Tumor markers decreased, tumors were shrank and decreased in size on the X-ray or CT scan measurement. The patients' lifestyle become manageable, and improved considerably. The treatment period were varied from 6 to 12 months. At this report in the treatment groups no patients expired. However, all patients in control group, who had not wish to be treated, succumbed to their related cancers in 6-12 months.

After blood samples were collected and allowed to stand for two hours, then centrifuged at 4000 rpm for 10 minutes. The superantants were separated and stored in a deep freeze before analysis. The chemistry analysis was carried out by blood chemistry analyzer to determine the changes in total cholesterol, HDL, LDL, triglycerides, liver function tests (such as SGOT, SGPT, G-GPT), renal function tests, and coagulation factors (PT, PTT). All results were tested with student t-test and Microsoft Excel-7.0 program. The results are depicted in the table below, which were all within normal limits.

| Group | TC | TRG | HDL | SGOT | SGPT | GGTP | WBC | Hb |
|---|---|---|---|---|---|---|---|---|
| Bromelain | 210.3 ± | 165.5 ± | 43.3 ± | 34.7 ± | 63.3 ± | 7.2 ± | 6.7 ± | 12.3 ± |
| | 30.2 mg/dl | 28.3 mg/dl | 22.2 mg/dl | 6.2 u/I | 5.6 u/I | 2.1 u/I) | 2.8 k/ul | 2.1 gm/dl |

In more detail, at the conclusion of the 6 months treatment period, the results are depicted as follows:

There were two breast cancer patients. One patient experienced left breast tumors shrinkage from 10.5 cm by 8.4 cm to 2.5 cm by 1.8 cm in size. The left axillary lymph node shrank from 2.5 cm by 1.6 cm to 0.5cm by 0.4 cm within 3 months of intravenous Bromelain therapy, and achieved complete remission in 6 months. The second patient's right breast tumor shrank from 6.5 cm by 4.8 cm to 2.8 cm by 1.5 cm. with 4 months of intravenous Bromelain therapy.

There were two lung cancer patients. One patient experienced the shrinkage from 4.6 cm by 5.1 cm to 1.8 cm by 1.2 cm with 12 weeks of intravenous Bromelain therapy with further therapy for 4 months the tumors were completely healed. Another patient with lung tumor of 3.9 cm by 4.5 cm experienced reduction of the tumor to 1.5 cm by 1.9 cm with 5 months of intravenous Bromelain therapy.

There were two colon cancer patients. Two patients with stage-IV disease and widely metastasis in the abdominal cavity were treated with intravenous Bromelain infusion for 12 months and now show no evidence of persistent disease.

There were two ovarian cancer patients. Both patients suffered stage-III cancer with wide intra-abdominal metastasis, both had been treated with intravenous Bromelain infusion for 12 months, and at this time showed no evidence of persistent disease on 2nd look laparotomy.

There were two patients with cervical cancer. Both patients were stage-IV with rectal and urinary bladder invasions. After intense Bromelain intravenous treatment for 12 months, there was no further evidence bladder or rectal invasion. No tumors could be detected.

There were two patients with Uterine cancer. Both patients were Stage-III with intravaginal metastasis. After intravenous Bromelain treatment for two months, tumors in the vagina showed necrosis and fibrosis with no evidence of persistent disease.

There were two patients comprising control group, who refused to be treated. One patient with cervical cancer stage-IV with cervical spine metastasis, and one patient with breast cancer stage-IV with pulmonary metastasis. Both patients refused treatment, and both succumbed to their diseases with pulmonary, and septic infections in 9 to 12 months after frequent in-hospital care and treatments.

There was case of Hepatoma (hepato-cellular carcinoma). This tumor when it was discovered it was 15 by 15 by 10 cm in size. The surgical resection was not feasible, he received one course of chemotherapy without result and suffered from septsis and bacteremia for 2 months, then he was given IV Bromelain therapy for 7 months. The patient then achieved complete remission.

Again, this is evidence that administration of Bromelain extract (Bromelainases) in humans could treat various types of cancer and neoplastic diseases. Liver and renal function, lipid profile, white blood cells and hemoglobin were not affected.

While the present invention and discovery has been described with respect to the above specific descriptions and embodiments, it should be recognized that various modifications and changes may be made to the present invention by those skills in the art which also fall within the scope of the invention as defined by the appended claims and their legal equivalents.

That which is claimed:

1. A composition of matter can reduce size of cancerous tumors in mammals, the composition of matter comprising
Bromelain infused with vitamin C creating a protein enzyme complex which can be administered in an effective amount to reduce the size of cancerous tumors in mammals.

2. The composition of matter of claim 1, further comprising
the effective amount is 50 to 60 mg of active ingredient per kilogram of body weight for the body weight of 50 to 60 kg; and
the effective amount is administered intravenously in a 3 to 4 hour period for 12 weeks to 2 years.

3. The composition of matter of claim 1, further comprising
500 mg of active ingredient are combined with 200 mg of the Vitamin C and 50 mg of a starch or lactose carrier to create a 750 mg capsule.

4. The composition of matter of claim 1, further comprising
1000 mg of active ingredient are combined with 300 mg of the Vitamin C and 200 mg of a starch or lactose carrier to create a 1500 mg capsule.

5. The composition of matter of claim 1, further comprising
1500 mg of active ingredient are combined with 300 mg of the Vitamin C and 100 mg of a starch or lactose carrier to create a 1900 mg capsule.
